(19) United States
(12) Patent Application Publication
(10) Pub. No.: US 2024/0105979 A1
(43) Pub. Date: Mar. 28, 2024

(54) BIOFUNCTIONAL ELECTRODE FOR STORING AND RELEASING COMPOUNDS BY SLOW DIFFUSION

(71) Applicants: CENTRE NATIONAL DE LA RECHERCHE SCIENTIFIQUE, Paris (FR); UNIVERSITE DE LORRAINE, Nancy (FR); UNIVERSITE GRENOBLE ALPES, Saint Martin d'Heres (FR)

(72) Inventors: Serge COSNIER, Crolles (FR); Stefan MC MURTRY, Laxou (FR); Omar EL MAZRIA, Seichamps (FR); Hanna RIAHI, Vandoeuvre les Nancy (FR); Yannig NEDELLEC, Grenoble (FR)

(21) Appl. No.: 18/255,919

(22) PCT Filed: Dec. 8, 2021
(86) PCT No.: PCT/EP2021/084815

§ 371 Date: Jun. 5, 2023

(30) Foreign Application Priority Data
Dec. 9, 2020 (EP) 20306523.0

(51) Int. Cl. H01M 8/16

(52) U.S. Cl. CPC H01M 8/16; H01M 2300/0005

(57) ABSTRACT

An electrode including a storage film. The storage film includes a layer of a support material including at least one inclusion in which a compound of interest is stored and a wall of permeable sealing material sealing the at least one inclusion. The storage film further includes a porous layer covering, at least in part, the wall of sealing material, at least a portion of a surface of the porous layer constituting an active surface of the electrode.

### TECHNICAL FIELD

The present invention relates to biofunctional devices. The present invention concerns, in particular but not exclusively, the field of analytical devices, such as (bio)sensors, (bio)electrochemical devices, such as (bio)batteries, and devices for release/liberation of (bio)chemical/biological compounds.

### STATE OF THE PRIOR ART

Biobatteries are known in the state of the prior art, the operation of which requires the addition of (bio)chemical products, such as for example additives, cofactors or enzyme substrates. In fact, the biobatteries of the state of the art require chemical compounds, for example the enzyme or microbial substrate consumed at the anode, to be added continuously so that the biobattery can function.

Biosensors are also known in the state of the prior art comprising a bioreceptor, for example proteinaceous or enzymatic. The operation of such biosensors is based on activation or inhibition of the bioreceptor. Activation of the bioreceptor allows, in most cases, a specific detection of a particular analyte, while inhibition of the bioreceptor allows, in most cases, detection of a group of inhibitor compounds.

Capsules or pills are known in the state of the prior art for release/liberation of compounds, mainly of medicinal compounds, in a controlled manner in particular in an organism. In the case of capsules, the majority of these are submillimetric in size and intended to be introduced directly into a liquid biological medium so as to migrate to a specific target of the organism.

An aim of the invention is in particular:
to propose a macroscopic sample for storing one or more compounds in order to liberate them later, and/or
to propose a macroscopic sample for preserving one or more compounds intended to be released later, and/or
to propose a macroscopic sample in order to liberate, at the appropriate time in a uniform, continuous and controlled manner, one or more compounds stored in the sample, and/or
to propose a macroscopic sample in the form of a film, having a thickness less than one millimetre, preferably less than 500 microns, which separates two mainly flat planes the surface area of which is greater than one square millimetre, preferably greater than one square centimetre, capable of liberating one or more compounds stored in the sample in a uniform, continuous and controlled manner, and/or
to propose an analytical device intended to be used directly on site and which does not require the addition of any external compound or element in order to function, and/or
to produce electrodes, in particular (bio)anodes, for (bio)batteries, intended to be used directly in any medium, for example a biological medium, and not requiring any addition of products or compounds in order to function, and/or
to produce biosensors, in particular enzyme biosensors, intended to be used directly on site and not requiring any addition of products or compounds in order to function.

### DISCLOSURE OF THE INVENTION

To this end, an electrode is proposed comprising:
a storage film comprising:
   a layer of a support material comprising at least one inclusion, the at least one inclusion containing a compound of interest,
   a wall of sealing material sealing the at least one inclusion, said wall of sealing material being capable of allowing the compound of interest to pass or diffuse therethrough,
a porous layer at least partially covering the wall of sealing material, at least a portion of a surface of the porous layer constituting an active surface area of the electrode.

The at least one inclusion can comprise one or more compounds of interest. The compound(s) of interest can be liquid or solid, for example a powder.

Preferably, the wall of sealing material is called permeable.

Preferably, the wall of sealing material is capable of enabling the compound of interest to pass or diffuse through the wall of sealing material.

Preferably, the permeable wall of sealing material is capable of allowing the compound of interest to pass through the wall of sealing material in a controlled manner, for example according to a desired speed of passage or diffusion.

A wall of the at least one inclusion can comprise, or can be constituted wholly or partially by, the wall of sealing material.

The at least one inclusion can have a minimum Feret diameter greater than 0.1 μm and a maximum Feret diameter less than the diameter of the inclusion.

Preferably, the inclusion is wholly comprised within the layer of support material. Preferably, the storage film encompasses or envelops the inclusion.

Preferably, the layer of support material and the wall of sealing material together form an assembly or a layer or a film all in one piece. The assembly or the layer or the film all in one piece can constitute the storage film.

The storage film can be a biofunctional device according to the invention.

The electrode can comprise a surface, called release or liberation surface, from which the compound of interest is released or liberated from the storage film, said release surface:
comprises, at least partially, a surface of the wall of sealing material,
is situated facing the porous layer.
   The porous layer can cover, at least partially, the release surface.

The release surface can comprise, at least partially, one of the faces or a surface of the storage film. One of the faces or the surface of the storage film can correspond to a portion of the total surface area of the storage film.

Preferably, the permeable wall of sealing material is capable of liberating or releasing the functional compound through the release surface.

Preferably, the permeable wall of sealing material is capable of allowing the compound of interest to pass through the wall of sealing material when, preferably only in the event that, the release surface is in contact with a predetermined medium.

In the present application, by “permeable wall” may be understood a wall, a film or a layer of material allowing the free diffusion of one or more species therethrough.

The wall of sealing material can be permeable due to:
the intrinsic property of the sealing material to allow the compound of interest to pass or diffuse through, or
the presence of at least one pore, or at least one perforation, in the sealing material; the sealing material being impermeable, i.e., intrinsically incapable of allowing the compound of interest to diffuse or pass through.

The wall of sealing material can be permeable, moreover, to one or more predetermined media in which the electrode(s) is(are) intended to be immersed.

Preferably, the permeable wall of sealing material is capable of liberating or releasing the functional compound through the release surface in a controlled manner, for example according to a desired speed of release or liberation. Preferably, the speed of release or of liberation is equal to a speed of passage or of diffusion of the compound of interest through the sealing material.

Preferably, the compound of interest is liberated/released from the release surface in a medium with which the release surface is intended to be placed in contact.

The porous layer can comprise a catalyst.

The catalyst can be inorganic, for example a coordination complex or compound, or organic, for example an enzyme catalyst.

According to the invention:
the sealing material or the sealing material and the support material can be conductive materials, or
the storage film can be insulating and/or the porous layer can comprise a porous conductive material and the catalyst.

The electrode can be intended to be used as electrode of a battery or of a biobattery, for example a biofuel cell. The electrode can be intended to be used as electrode of a biobattery or as electrode of a biosensor.

The catalyst can be immobilized in and/or on and/or under the porous conductive material.

The porous layer can also comprise a permeable trapping material in which the catalyst is contained.

The permeable trapping material is capable of allowing the compound of interest to pass through, preferably in a controlled manner. The permeable trapping material is capable of allowing ions to pass through, preferably an electrolyte or ions capable of constituting an electrolyte.

The trapping material can be an electrical insulator.

The trapping material can comprise an oxygen reduction mediator, called redox mediator, capable of enabling an electric current to pass within the trapping material. In this case, the porous layer can be a porous conductive layer.

The trapping material can be a gel and/or a polymer. Preferably, the catalyst is immobilized in the trapping material.

The trapping material can form a layer or a film or can be an assembly of nano- and/or micro- and/or macroparticles. The film or the layer of trapping material can be disposed in and/or on and/or under the porous conductive material. The assembly of particles forming the trapping material can be distributed within the porous layer or can be amassed in a zone of the porous layer.

The at least one inclusion can have the form of a cavity extending in the layer of the support material.

The electrode can comprise several individual inclusions each having an oblong or round shape extending mainly within the thickness (e) of the storage film.

Preferably, the thickness (e) of the storage film extends mainly in a direction connecting the storage film to the porous layer.

The at least one inclusion can constitute at least one individual reservoir enveloped, at least partially, in the layer of the support material.

The storage film can comprise an encapsulating material enveloping at least partially the at least one inclusion such that a surface of the encapsulating material constitutes at least a portion of a wall of the at least one inclusion.

Preferably, the encapsulating material constitutes a film or a layer. Also preferably, the encapsulating material envelops the inclusion only partially.

The encapsulating material can be constituted by a material different from the support material. The encapsulating material can be identical to the support material.

The encapsulating material can be identical to the sealing material. Preferably, the layer of support material and the film of encapsulating material form an assembly or a layer or a film all in one piece. The assembly, the layer or the film all in one piece formed by the layer of support material and the film of encapsulating material can constitute the layer of support material.

Preferably, the layer of support material, the film of encapsulating material and the wall of sealing material form an assembly or a layer or a film all in one piece. The assembly, the layer or the film all in one piece formed by the layer of support material, the film of encapsulating material and the wall of sealing material can constitute the storage film.

Preferably, the support material, the encapsulating material and/or the sealing material are flexible and/or resilient.

Preferably, the support material, the encapsulating material and/or the sealing material is a polymer material.

The sealing material can be different from the support material and/or from the encapsulating material.

The wall of sealing material can be a porous or semi-permeable wall.

By “porous wall” may be understood a layer, a film or a wall comprising at least one pore. The pore can be a duct or channel, passing through, from one side to the other, the wall in which it extends.

A pore diameter or a diffusion coefficient can be arranged to obtain a speed of release or of liberation of the desired compound of interest. A speed of release or of liberation of the compound of interest can be a function of the medium in which the electrode is intended to be immersed.

The sealing material can be porous or permeable or semi-permeable.

The sealing material can be impermeable and can comprise one or more pores arranged in the wall to render permeable the wall of sealing material.

According to the invention, use of the electrode according to the invention as a battery or biobattery or in a battery or a biobattery is also proposed.

According to the invention, use of the electrode according to the invention as a sensor or biosensor or in a sensor or a biosensor is also proposed.

According to the invention, use of the electrode according to the invention as a biofunctional device or in a biofunctional device is also proposed.

According to the invention, a method for the manufacture of an electrode comprising a storage film is also proposed, said storage film comprising at least one inclusion, said method comprising the steps consisting of:
forming at least one cavity in a layer of a support material,
depositing a film of sealing material on the layer of support material to seal the at least one cavity so as to form the at least one inclusion in the layer of support material,
forming a porous layer on the film of sealing material; at least a portion of a surface of the porous layer being capable of forming an electrode active surface area of the electrode.

Preferably, the porous layer comprises the porous conductive material and the catalyst.

The film of sealing material can be applied on the layer of support material.

Preferably, the at least one inclusion comprises the compound of interest. The compound of interest in powder form can be intended to be released or liberated by dipping the electrode in a liquid medium. A compound of interest in powder form can have the advantage of being released more slowly than a compound of interest stored in liquid form.

The compound of interest can be released or liberated, in a controlled manner, for example when the electrode is immersed in a predetermined liquid. The compound of interest can be released or liberated, preferably only, when the electrode is immersed in a predetermined liquid.

The at least one cavity has a minimum Feret diameter greater than 0.1 μm and a maximum Feret diameter less than the diameter of the inclusion.

By “film of sealing material” may be understood a solid film, a solid strip or a solid layer of material that is deposited on the layer of support material. Preferably, the film of sealing material is all in one piece.

Preferably, each cavity is comprised within the layer of support material.

Preferably, each cavity contains a single aperture to be sealed.

Each aperture of each cavity can be sealed by the wall of sealing material.

Preferably, after the step of deposition of the film of sealing material, the inclusion is sealed by the film of sealing material which constitutes the wall of sealing material.

The wall of sealing material can be permeable.

The method can comprise:
prior to the step of formation of the at least one inclusion, a step consisting of depositing the layer of support material on a substrate layer,
after the step of deposition of the film of sealing material, a step consisting of dissolving the substrate layer so as to obtain the storage film.

The method can comprise, prior to the step of deposition of the film of sealing material, a step consisting of covering a wall of the at least one cavity formed in the layer of support material with a layer of an encapsulating material.

The wall of the at least one cavity can correspond to the wall of the at least one inclusion, with the exception of the portion of the wall of the at least one inclusion formed by the sealing material, when the step of covering the wall of the at least one cavity is not implemented.

The wall of the at least one cavity can be different from the wall of the at least one inclusion when the step of covering the wall of the at least one cavity is implemented. In this case, the encapsulating material can constitute the wall of the at least one inclusion.

The step consisting of covering a wall of the at least one cavity can comprise deposition, preferably isotropic, of a layer or a film of encapsulating material.

The method can comprise a step consisting of filling the at least one inclusion with a compound of interest.

The step of filling the at least one cavity can be implemented prior to the step of deposition of the film of sealing material.

Implementation of the filling step prior to the deposition step is particularly suitable for solid compounds of interest but can also be suitable for liquid compounds of interest.

The step of filling the at least one occlusion can be implemented, after the step of deposition of the film of sealing material, by injecting the compound of interest or a precursor of the compound of interest, by means of a capillary tube or a needle passing through the wall of sealing material.

Implementation of the filling step after the deposition step is particularly suitable for liquid compounds of interest but can also be suitable for solid compounds of interest.

After the deposition step, the method can comprise a step consisting of rendering permeable the wall of sealing material by mechanical perforation or by ultrasound, chemical, optical or plasma treatment of the sealing wall.

The method can comprise a step consisting of producing one or more perforations in the wall of sealing material; the perforation(s) having a diameter greater than 10 nm.

The filling step can be implemented concomitantly with the perforation step when the filling step is carried out by injection of the compound of interest or of the precursor of the compound of interest by means of a capillary tube or of a needle passing through the wall of sealing material.

Preferably, the perforation step is implemented prior to the step consisting of forming a porous layer on the film of sealing material.

The diameter of the perforation(s) can extend in a direction perpendicular to the thickness of the wall of sealing material. The thickness of the wall of sealing material can extend in the direction connecting the storage film to the porous layer.

The step of deposition of the porous layer can comprise the sub-steps consisting of:
depositing a porous conductive material on at least a portion of the wall of sealing material, then
immobilizing a catalyst (8) in and/or on and/or on the porous conductive material;
   the porous layer comprising the porous conductive material and the catalyst.

Preferably, the porous conductive material is deposited on at least a portion, preferably the whole, of the release surface.

The method can comprise a step consisting of trapping and/or immobilizing the catalyst in a trapping material.

After, or concomitantly with, the step of trapping the catalyst in the trapping material, the method can comprise a step consisting of depositing the trapping material containing the catalyst in and/or on and/or under the porous conductive material.

The method according to the invention is suitable for implementation of the electrode according to the invention. The electrode according to the invention is, preferably, implemented by the method according to the invention. Preferably, the method according to the invention is specially designed to implement the electrode according to the invention. Thus, any characteristic of the method according to the invention can be integrated in the electrode according to the invention and vice versa.

### DESCRIPTION OF THE FIGURES

Other advantages and features of the invention will become apparent from reading the detailed description of implementations and embodiments that are in no way limitative, and from the following attached drawings:

FIGS. 1A, 1B and 1C are diagrammatic representations of the steps of the method for manufacturing biofunctional devices according to the invention,

FIGS. 2A, 2B, 2C and 2D are diagrammatic representations of the steps of the method for manufacturing biofunctional devices according to the invention,

FIGS. 3A and 3B are diagrammatic representations of electrodes according to the invention,

FIG. 4 is a graph showing the development of the variation in the molar concentration of methylene blue, liberated through the release surface, as a function of time for two biofunctional devices according to the invention: a storage film and an electrode,

FIG. 5 is a graph showing the development of the cathode current, generated by the reduction of ortho-quinone to catechol by the electrode according to the invention, as a function of time and of the quantity of dissolved oxygen in solution; the electrode, comprising tyrosinase as catalyst and catechol as compound of interest, is used as a biosensor for detecting dissolved oxygen in solution,

FIG. 6 is a graph showing the development of the cathode current, generated by the reduction of ortho-quinone to catechol by the electrode according to the invention, as a function of time and of the quantity of benzoic acid in solution; the electrode, comprising tyrosinase as catalyst and catechol as compound of interest, is used as a biosensor for detecting benzoic acid in solution.

### DESCRIPTION OF THE EMBODIMENTS

As the embodiments described hereinafter are in no way limitative, variants of the invention can be considered in particular comprising only a selection of the characteristics described, in isolation from the other characteristics described (even if this selection is isolated within a phrase comprising these other characteristics), if this selection of characteristics is sufficient to confer a technical advantage or to differentiate the invention with respect to the state of the prior art. This selection comprises at least one, preferably functional, characteristic without structural details, or with only a part of the structural details if this part alone is sufficient to confer a technical advantage or to differentiate the invention with respect to the state of the prior art.

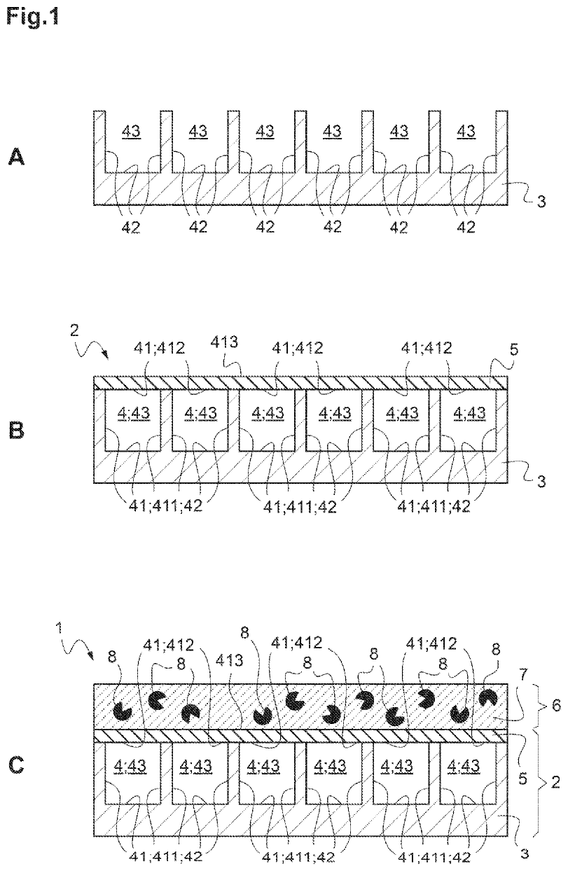

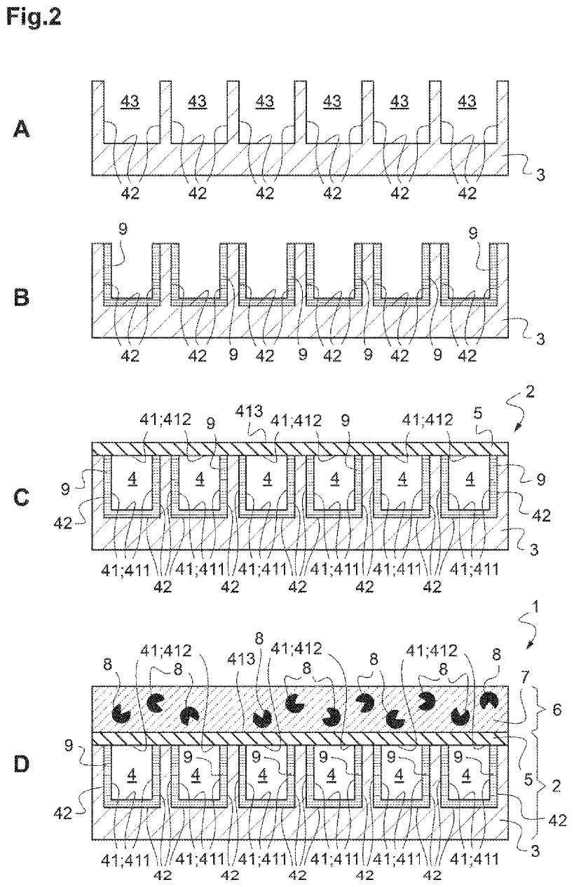

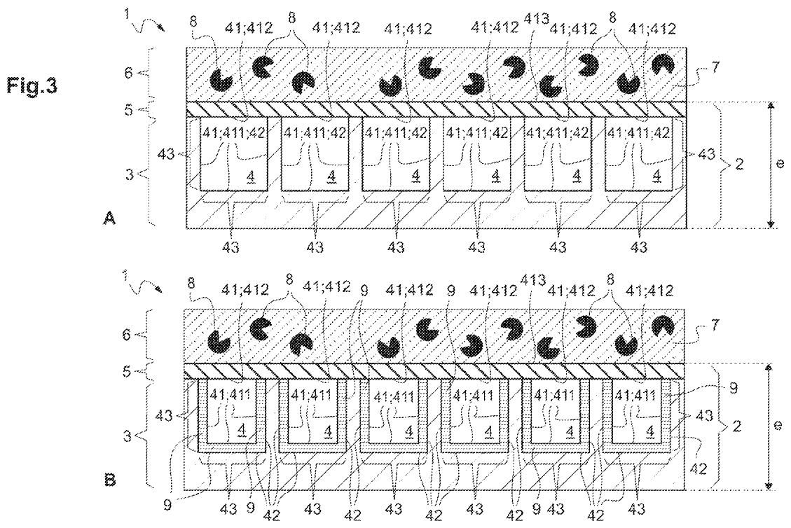

With reference to FIGS. 1 and 2, an embodiment of a method for the manufacture of a biofunctional device 1 is shown. With reference to FIGS. 3, an embodiment of an electrode 1 is shown. The thickness of the storage film (2) is typically comprised between 50 microns and several centimetres. Preferably, the thickness of the storage film (2) is comprised between 500 μm and 5 mm. The storage film 2 comprises at least one inclusion 4.

With reference to FIGS. 1 and 2, the method comprises the step consisting of forming at least one cavity 43 in the layer 3 of support material. The support material is a polymer. By way of non-limitative example, the support material is a flexible (deformable) or rigid (non-deformable) material such as silicon, zinc oxide, aluminium nitride, silicon oxide, aluminium oxide, polymers, plastic materials and metals. Preferably, the support material is polyurethane (PU) or polymethyl methacrylate (PMMA). The at least one cavity 43 has a Feret diameter comprised between 0.05 μm and 5 mm, preferably between 0.1 μm and 1 mm.

The manufacturing method also comprises the step consisting of depositing the film 5 of sealing material on the layer 3 of support material to seal the at least one cavity 43 so as to form the at least one inclusion 4 in the layer 3 of support material. By way of non-limitative example, the sealing material is polyurethane (PU) and therefore impermeable according to the embodiment. The thickness of the film 5 of sealing material is typically comprised between 1 μm and 1 cm, preferably between 25 μm and 400 μm. The sealing material can be selected from the metallic materials, semiconductor materials, piezoelectric materials, insulating materials and polymers such as epoxy resins and biocompatible resins. By way of non-limitative example, the sealing material can be PMMA, polyurethane, biocompatible polymers, metal, such as gold for example. The sealing material is capable of withstanding the solvent used during the step of dissolving the resin.

The method comprises a step consisting of filling the at least one inclusion 4 with a compound of interest that depends on the type of application envisaged. The step of

filling the at least one cavity 43 is implemented prior to the step of deposition of the film 5 of sealing material. The filling step consists of immersing the storage film 2, prior to the step of deposition of the film 5 of sealing material, in a compound of interest solution. Alternatively, the filling step can consist, prior to the step of deposition of the film 5 of sealing material, of pouring the compound of interest, for example in powder form, into the inclusion 4.

The method comprises a step consisting of producing one or more perforations in the wall 5 of sealing material. According to an embodiment, the perforation(s) are produced by means of a capillary tube or a needle by mechanical perforation of the wall (5) of sealing material. The perforation(s) have a diameter greater than 10 nm. By way of non-limitative example, the diameter of the perforation(s) is comprised between 100 μm and the Feret diameter of the cavity 43 according to the application envisaged as well as according to the type and solid or liquid state of the compound of interest.

According to a non-limitative embodiment, the method comprises the step consisting of forming a porous layer 6 on the film 5 of sealing material. The thickness of the porous layer 6 is typically comprised between 400 nm and 20 μm. Preferably, the thickness of the porous layer 6 is typically comprised between 1 and 5 μm. At least a portion of a surface of the porous layer 6 is capable of forming an active surface area of the electrode 1.

The step of deposition of the porous layer 6 comprises the step consisting of depositing a porous conductive material 7, carbon nanotubes (CNT) 7 according to the embodiment, on at least a portion of the wall 5 of sealing material, on the whole of the sealing wall 5 according to the embodiment. The thickness of the CNT 7 is typically identical to the thickness of the porous layer 6. The step of deposition of the porous layer 6 also comprises the step consisting of immobilizing a catalyst 8 in and/or on the CNTs 7. The porous layer 6 therefore comprises the CNTs 7 and the catalyst 8.

According to the non-limitative embodiment, and with reference to FIG. 2B, the method comprises, prior to the step of deposition of the film 5 of sealing material, a step consisting of covering a wall 42 of the at least one cavity 43, formed in the layer 3 of support material with a layer 9 of an encapsulating material of PU. In this case, the wall 41 of the layer 9 of the encapsulating material constitutes the wall 41 of the inclusion 4 with the exception of the portion 412 of the wall 41 of the at least one inclusion 4 formed by the sealing material. The thickness of the layer 9 of encapsulating material is typically greater than 10 nm, preferably comprised between 30 μm and half of the Feret diameter of the cavity 43. The encapsulating material can be selected from metallic materials, semiconductor materials, piezoelectric materials, insulating materials and polymers such as epoxy resins and biocompatible resins. Preferably, the encapsulating material is capable of withstanding the solvent used in the step of dissolving the resin. By way of non-limitative example, the encapsulating material can be PMMA, polyurethane, biocompatible polymers, metal, such as gold for example.

With reference to FIGS. 3, an electrode 1 is described comprising a storage film 2 and a porous layer 6. The storage film 2 comprises a layer 3 of a support material comprising at least one inclusion 4. The at least one inclusion 4 contains at least one compound of interest that differs according to the type of application envisaged. The storage film 2 also comprises a wall 5 of permeable sealing material sealing the at least one inclusion 4. The wall 5 of permeable sealing material is a porous wall 5.

According to the embodiment, the electrode 1 is obtained directly by implementation of the method described above.

The at least one inclusion 4 constitutes at least one individual reservoir 4 enveloped, at least partially, in the layer 3 of the support material.

According to the embodiment, the electrode 1 comprises several individual inclusions 4 each having an oblong shape and extending mainly within the thickness e of the storage film 2.

The porous layer 6 covers, at least partially, wholly according to the embodiment, the wall 5 of sealing material. At least a portion of a surface of the porous layer 6 constitutes an active surface area of the electrode 1.

According to the embodiment, the wall 41 of the at least one inclusion 4, with the exception of the portion 412 of the wall 41 of the at least one inclusion 4 formed by the sealing material, is a surface of the support material.

The electrode 1 comprises a release surface 413 from which the compound of interest is liberated from the storage film 2. The release surface 413 comprises, at least partially, a surface of the wall 5 of sealing material. The release surface 413 is situated facing the porous layer 6. According to the embodiment, the surfaces of the wall 5 of sealing material situated facing the porous layer 6 and opposite the portions 412 of the wall 41 of the sealing material forming portions 412 of wall 41 of the inclusions 4 constitute the release surface 413.

According to a non-limitative embodiment, the support material is identical to the sealing material. The storage film 2 is therefore insulating according to the embodiment.

The porous layer 6 comprises a catalyst 8, an electrocatalyst 8 according to the embodiment. The porous layer 6 also comprises a porous conductive material 7, carbon nanotubes (CNT) 7 according to the embodiment. According to the embodiment, at least a portion of the CNTs 7 constitutes the active surface area of the electrode 1.

According to a non-limitative embodiment shown in FIG. 3B, the storage film 2 comprises an encapsulating material 9 enveloping at least partially the at least one inclusion 4 such that a surface 411 of the encapsulating material constitutes at least a portion of the wall 41 of the at least one inclusion 4.

In this case the wall of the at least one inclusion 4, with the exception of the portion 412 of the wall 41 of the at least one inclusion 4 formed by the sealing material, is a surface of the film 9 or of the layer 9 of encapsulating material enveloping the inclusion 4.

According to this non-limitative embodiment, the encapsulating material 9 is PU.

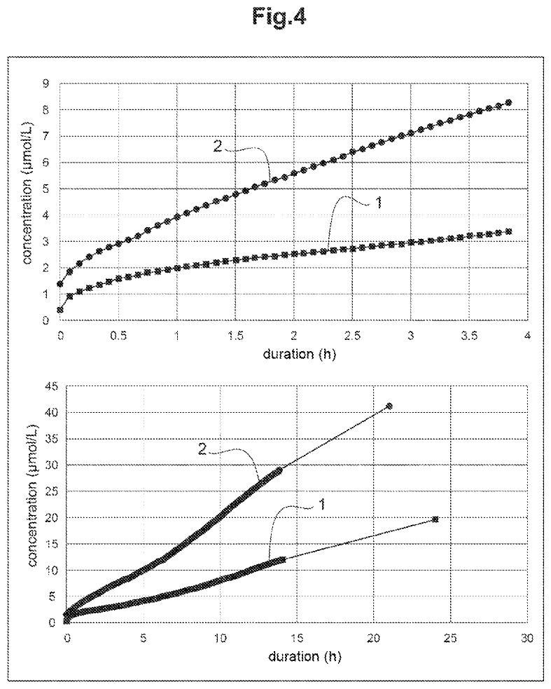

With reference to FIG. 4, a graph is shown presenting the variation in the molar concentration of a compound of interest in a solution in which an electrode 1 and a storage film 2 according to the invention is immersed. The compound of interest is methylene blue which is liberated by the storage film 2 in the solution as a function of time. The speed of release and the regularity of the release were evaluated for the storage film 2 according to the invention and for the electrode 1 according to the invention.

The methylene blue is stored in powder form in the inclusions 4. The step of filling the inclusions 4 was implemented by flow of the methylene blue in powder form into the inclusions 4 under the effect of gravity. The perforation step was implemented by producing, for each inclusion 4, a single perforation of the portion 412 of the wall 41 of each inclusion 4 formed by the sealing material by means of a needle of a diameter typically equal to 100 μm. The perforation step was carried out using an optical microscope and a mechanical guidance system of the needle.

With respect to the electrode 1, the step of depositing the CNTs 7 was carried out by deposition of a solution (dispersion) of CNT 7 on the wall 5 of sealing material of a storage film 2 according to the invention. The solution was left for 120 minutes under vacuum on the wall 5 then rinsed. The concentration of CNT 7 in the solution of CNT 7 is 5 mg/ml. The solvent is ethylene glycol. Solvents such as N,N-dimethylformamide (DMF) or N-methylpyrrolidone (NMP) were proscribed because they denature and damage most polymers and therefore the storage film 2 according to the embodiment. After rinsing the storage film 2, the CNTs 7 adhere strongly to the storage film 2, the electrode 1 thus formed is ready for use.

The storage film 5 was immersed in a volume of 2.5 ml of aqueous solution. The concentration of methylene blue dissolved in the aqueous solution was measured by UV/visible spectrometry. FIGS. 4a and 4b show the development of the total concentration of methylene blue in micromoles per litre (μmol/l) in the aqueous solution as a function of time in hours (h). A linear increase in the concentration is observed over time. This indicates a constant speed of release. The speed of release of the storage film 2 is comprised between 1.5 and 4 μmol/l/h. The speed of release of the electrode 1 is comprised between 0.5 and 0.8 μmol/l/h.

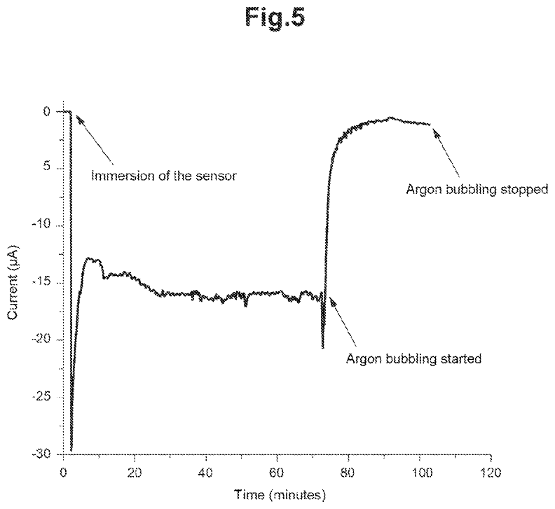

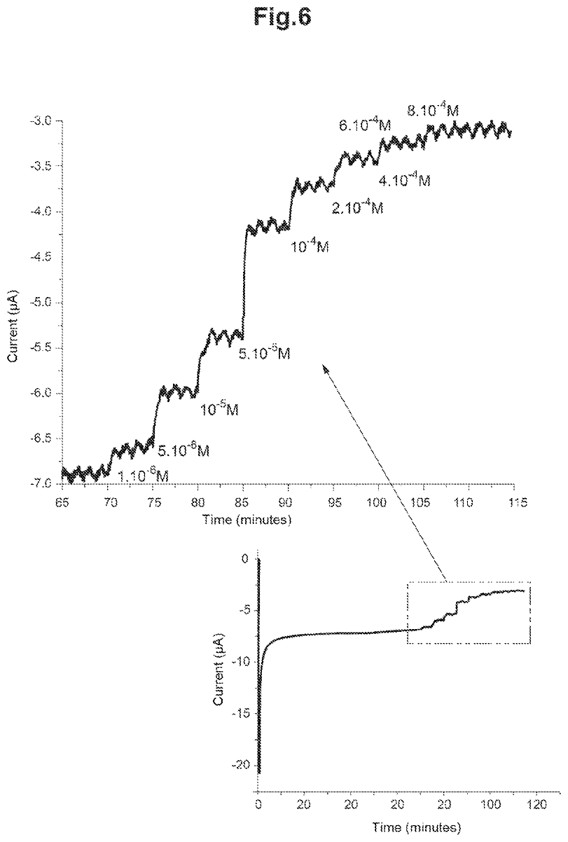

With reference to FIGS. 5 and 6, a use as biosensor of the electrode as shown in FIG. 3 is described. According to the embodiment in question, the porous layer 6 comprises CNTs 7, an enzyme catalyst 8, tyrosinase 8, and a clay, laponite. The compound of interest stored in the inclusion 4, which is liberated progressively during use of the electrode 1, is catechol. The filling step was implemented by flow into the inclusions 4 under the effect of gravity, of catechol in powder form. The perforation step was implemented by producing, for each of the two inclusions 4, a single perforation of the portion 412 of the wall 41 of each inclusion 4 formed by the sealing material by means of a needle of a diameter typically equal to 100 μm.

In practice, the method of implementation of the porous layer 6 comprises dispersion and delamination of laponite in aqueous medium. Laponite is present at a mass concentration of 1 mg/ml. Tyrosinase 8 is then mixed with the aqueous solution of laponite at a concentration of 10 mg/ml. The solution comprising the laponite-tyrosinase mixture is then deposited on the electrode 1, comprising a CNT film 7 on the wall 5 of sealing material of the storage film 2. The CNTs 7 were deposited beforehand on the electrode 1, according to the description above. The volume of laponite-tyrosinase solution deposited is such that approximately 300 μg of tyrosinase 8 and 30 μg of laponite lies on the film of CNT 7. After deposition of the solution comprising the laponite-tyrosinase mixture, evaporation of the solvent, i.e., water, is then carried out under vacuum. The electrode 1 is then placed under glutaraldehyde vapour for 45 min in order to cross-link the laponite-tyrosinase mixture. Before use of the electrode 1, the laponite is gelled by dipping the electrode 1 in a hydrogen phosphate/dihydrogen phosphate buffer solution for 20 min.

With reference to FIGS. 5 and 6, the development of the cathode current generated by the electrode used as biosensor 1 is shown. For conducting the experiment, the electrode 1 is immersed in a hydrogen phosphate/dihydrogen phosphate buffer solution, called buffer solution. In the presence of oxygen and catechol, the tyrosinase 8 oxidizes the catechol, which is liberated at the level of the release surface 413, as ortho-quinone. By applying a voltage of −0.2 Volts, with respect to the reference potential of the saturated calomel electrode (SCE), at the electrode 1, the appearance of a cathode current generated at the electrode 1 is noted.

With reference to FIG. 5, the possibility of detecting the presence or absence of dissolved oxygen in solution by means of the electrode 1 is shown. By means of prior calibration, it is also possible to quantify the quantity of dissolved oxygen in solution. The cathode current generated at the electrode 1 stabilizes at approximately 15 μamperes (μA) after around twenty minutes. After 70 minutes, argon is bubbled into the buffer solution. This has the effect of removing almost all of the dissolved oxygen in the buffer solution and therefore substantially reducing, or even stopping, the tyrosinase 8 activity and therefore the oxidation of the catechol by the tyrosinase 8. The quantity of ortho-quinone in the porous layer 6 therefore also reduces when the concentration of dissolved oxygen reduces. A drop in the cathode current to a value close to 0 Amperes is then noted. The drop in the current is proportional to the reduction in the oxygen concentration in solution. This demonstrates that the cathode current generated at the electrode 1 indeed originates from the reduction of ortho-quinone to catechol by the electrode 1.

With reference to FIG. 6, the possibility of detecting the presence of inhibitors of tyrosinase 8, here benzoic acid, by means of the electrode 1 is shown. This inhibitor can be a pollutant. By means of prior calibration, it is also possible to quantify the quantity of benzoic acid in solution. The cathode current generated at the electrode 1 stabilizes at approximately 7.5 μA after around twenty minutes. After 65 minutes, nine successive injections of known quantities of benzoic acid into the buffer solution are carried out. This has the effect of progressively inhibiting the tyrosinase 8, which therefore sees its activity reduce when the benzoic acid concentration increases. The quantity of ortho-quinone in the porous layer 6 therefore also reduces when the concentration of benzoic acid increases. A stepped reduction of the cathode current generated at the electrode 1 is noted for each of the injections of benzoic acid. The current reduces from 7 μA, the value of the cathode current before the first injection carried out at the 65^{th} minute for a benzoic acid concentration of 0 Molar (M) up to 3 μA at the 112^{th} minute for a total benzoic acid concentration of 8.10⁻⁴ M. The current drop is proportional to the quantity of inhibitor in solution. This demonstrates that the cathode current generated at the electrode 1 indeed originates from the reduction of ortho-quinone to catechol by the electrode 1.

Thus, in variants of the above-described embodiments that can be combined together:
the sealing material is a permeable material, and/or
the wall 5 of sealing material is a semi-permeable wall 5, and/or
the sealing material or the sealing material and the support material are conductive materials, and/or
the thickness of the porous conductive material 7 is equal to the thickness of the porous layer 6 when the catalyst 8 and the porous conductive material 7 are deposited concomitantly, and/or
the substrate layer is polyvinyl acetate (PVA) or PMMA, and/or
the support material is different from the sealing material, and/or
the electrode 1 comprises several individual inclusions 4 each having a round shape, and/or
the method comprises:
   prior to the step of formation of the at least one inclusion 4, a step consisting of depositing the layer 3 of support material on a substrate layer,
   after the step of deposition of the film 5 of sealing material, a step consisting of dissolving the substrate layer so as to obtain the storage film 2, and/or
the step of filling the at least one occlusion 4 is implemented, after the step of deposition of the film 5 of sealing material, by injecting the compound of interest or a precursor of the compound of interest by means of a capillary tube or a needle passing through the wall 5 of sealing material, and/or
when the method does not comprise a step of covering the cavity 43 by a layer 9 of encapsulating material, the wall 42 of the cavity 43 constitutes the wall 41 of the inclusion 4, and/or
the perforation of the portion 412 of the wall 41 of each inclusion 4 formed by the sealing material is carried out by means of a needle having a diameter greater than 100 μm, and/or
the perforation step is implemented by a technique that does not generate a local joule effect, so as not to affect the wall of sealing material or introduce imprecision with respect to the size of the pores created, and/or
the perforation step is implemented by femto laser or by ultrasonic abrasion.

In addition, the different characteristics, forms, variants and embodiments of the invention can be combined together in various combinations to the extent that they are not incompatible or mutually exclusive.

1. An electrode, comprising:
a storage film comprising:
a layer of a support material comprising at least one inclusion, the at least one inclusion:
contains a compound of interest;
constitutes at least one individual reservoir enveloped, at least partially, in the layer of the support material;
has the form of a cavity extending in the layer of the support material;
a wall of sealing material sealing the at least one inclusion, said wall of sealing material configured for allowing the compound of interest to pass or diffuse therethrough in a controlled manner, for example according to a desired speed of passage or diffusion; and
a porous layer at least partially covering the wall of sealing material, at least a portion of a surface of the porous layer constituting an active surface area of the electrode.

2. The electrode according to claim 1, comprising a surface, called release or liberation surface, from which the compound of interest is released or liberated from the storage film, said release surface:
comprises, at least partially, a surface of the wall of sealing material,
is situated facing the porous layer.

3. The electrode according to claim 1, in which the porous layer comprises a catalyst.

4. The electrode according to any one of the preceding claim 1, in which:
the sealing material or the sealing material and the support material are conductive materials, or
the storage film is insulating and the porous layer comprises a porous conductive material and the catalyst.

5. The electrode according to claim 1, comprising several individual inclusions each having an oblong or round shape extending mainly within the thickness of the storage film.

6. The electrode according to claim 1, in which the storage film comprises an encapsulating material
at least partially enveloping the at least one inclusion so that a surface of the encapsulating material constitutes at least a portion of a wall of the at least one inclusion.

7. The electrode according to claim 1, in which the wall of sealing material is a porous or semi-permeable wall.

8. A use of the electrode according to claim 1, in a battery or in a sensor.

9. A method for the manufacture of an electrode comprising a storage film, said storage film comprising at least one inclusion, said method comprising the steps consisting of:
forming at least one cavity in a layer of a support material;
depositing a film of sealing material on the layer of support material to seal the at least one cavity so as to form the at least one inclusion in the layer of support material, said wall of sealing material being capable of allowing the compound of interest to pass or diffuse therethrough in a controlled manner, for example according to a desired speed of passage or diffusion, and the at least one inclusion:
contains a compound of interest;
constitutes at least one individual reservoir enveloped, at least partially, in the layer of the support material;
has the form of a cavity extending in the layer of the support material; and
forming a porous layer on the film of sealing material; at least a portion of a surface of the porous layer being capable of forming an electrode active surface area.

10. The method according to claim 9, comprising:
prior to the step of formation of the at least one inclusion, a step consisting of depositing the layer of support material on a substrate layer;
after the step of deposition of the film of sealing material, a step consisting of dissolving the substrate layer so as to obtain the storage film.

11. The method according to claim 9, comprising, prior to the step of deposition of the film of sealing material, a step consisting of covering a wall of the at least one cavity, formed in the layer of support material with a layer of an encapsulating material.

12. The method according to claim 9, comprising a step consisting of filling the at least one inclusion with a compound of interest.

13. The method according to claim 12, in which the step of filling the at least one cavity is implemented prior to the step of deposition of the film of sealing material.

14. The method according to claim 12, in which the step of filling the at least one occlusion is implemented, after the step of deposition of the film of sealing material, by injecting the compound of interest or a precursor of the compound of interest by means of a capillary tube or a needle passing through a wall of sealing material.

15. The method according to claim 9, comprising a step consisting of producing one or more perforations in the wall of sealing material; the perforation(s) having a diameter greater than 10 nm.

16. The method according to claim 9, comprising the step consisting of forming a porous layer on the film of sealing material; at least a portion of a surface of the porous layer being capable of forming an active surface area of the electrode.

17. The method according to claim 9, in which the step of deposition of the porous layer comprises the sub-steps consisting of:
depositing a porous conductive material on at least a portion of the wall of sealing material, then
immobilizing a catalyst in and/or on and/or under the porous conductive material;
the porous layer comprising the porous conductive material and the catalyst.
